(19) United States
(12) Patent Application Publication
(10) Pub. No.: US 2023/0355949 A1
(43) Pub. Date: Nov. 9, 2023

(54) DISINFECTING AND STERILIZING CAP ASSEMBLY AND RELATED METHODS

(71) Applicant: Murtaza Amir, Cumberland, MD (US)

(72) Inventors: Max Arocha, Plantation, FL (US); Murtaza Amir, Cumberland, MD (US)

(73) Assignee: Murtaza Amir, Cumberland, MD (US)

(21) Appl. No.: 18/245,536

(22) PCT Filed: Sep. 15, 2021
(86) PCT No.: PCT/US2021/050548

§ 371 Date: Mar. 15, 2023

Related U.S. Application Data
(60) Provisional application No. 63/078,798, filed on Sep. 15, 2020.

(51) Int. Cl. A61M 39/18; A61M 39/20

(52) U.S. Cl. CPC A61M 39/18; A61M 39/20

(57) ABSTRACT

A disinfecting cap assembly device for protecting disinfecting and sterilizing medical connectors, and methods for using the disinfecting and sterilizing cap assembly device to protect the cleanliness of medical connectors such as IV access ports or other types of devices having a Luer connections.

### BACKGROUND OF THE INVENTION

The present invention relates to a device and method for protecting and disinfecting an injection port. More specifically, the invention relates to device which is a cap assembly and a method employing the cap assembly to maintain a clean and substantially microbe-free or substantially sterile surface (providing a sterile field) on an injection port having a Luer-type fitting or connector, such as an injection port provided on an intravenous (IV) administration set.

The cap assembly can also be configured or adapted to couple to a stoppered vial containing sterile drug or diluent, maintaining a sterile field on an outer surface of the puncturable stopper. A vial containing sterile drug or diluent is generally designated for single-use only, whereby the vial and contents are disposed following a single puncture of the stopper using a hypodermic needle. By coupling a cap assembly of the subject invention to the stoppered end of the vial, a sterile field can be maintained under the cap assembly. This protection of the outer surface of the vial stopper can allow for a user to perform multiple punctures of the stopper with minimal microbial contamination to the stopper surface and the contents of the vial. Advantageously, this use of the cap assembly of the invention can effectively convert a single-use stoppered vial to a multi-use stoppered vial.

A Luer-type fitting is a standardized system of small-scale fluid fittings used for making leak-free connections between a male-taper fitting and its mated female part on medical and laboratory instruments, including hypodermic syringe tips, stopcocks, injection ports and hypodermic needles. Currently ISO 80369 governs the Luer standards and testing methods.

There are two varieties of Luer-type connections: locking and slipping. Reference to Luer lock fittings used herein means either or both types of Luer lock fittings, and includes “one piece Luer lock,” “two piece Luer lock” or “rotating collar Luer lock,” any of which are used interchangeably to refer to Luer-type connections in accordance with the subject invention.

Intravenous injection catheters (IV administration sets) having injection ports configured with Luer lock connectors for connecting a syringe or other compatible device for intravenous delivery of medication or removal of a blood sample from a patient are well known. When an injection port of an IV administration set is not in use, it is desirable to maintain the injection port surface clean and free from bacteria or other microbial contamination. Without protection from contamination, pathogenic microbes present on the exposed surface of the IV catheter, including the outer surface of the injection port, can be transferred to the patient by way of a needle or syringe being connected to the injection port during use, and can potentially cause infection.

Despite Guidelines issued by the Centers for Disease Control and Prevention, catheter-related bloodstream infections continue, and result in infections that can be a major cause of illness and excess medical costs. Approximately 80,000 catheter-related bloodstream infections occur in U.S. intensive care units each year. In addition to the monetary costs, these infections are associated with anywhere from 2,400 to 20,000 deaths per year. Accordingly, maintaining a clean and microbe-free or near sterile injection port can minimize the risk of infection.

To address this risk of infection, injection ports have been configured to have antimicrobial coatings on the intraluminal surfaces. However, there are various drawbacks to using antimicrobial coatings on the intraluminal surfaces of ports, including toxicity, development of microbial resistance, and increased costs of manufacture.

Another approach used to maintain the sterility of an injection port is through the use of caps. Currently, there are various products available for capping an injection port of an intravenous catheter or infusion set. These include caps having an antimicrobial solution which contacts the exterior surface of the injection port when the cap is positioned to cover the port. For example, as described in U.S. Pat. No. 8,343,525, certain threaded caps employ an alcohol-soaked material that is disposed within the cavity of the cap so that the material scrubs the exterior surfaces of the port when the cap is screwed on. However, these caps are required to be removed and separated from the injection port when giving an injection or withdrawing a sample from the injection port and therefore can allow the port to be exposed and contaminated. These removable caps can also be easily misplaced or lost, rendering them useless for their intended purpose. Tethered caps have been employed to minimize this problem of losing or misplacing the cap, but are still not useful if the user is not diligent in replacing the cap immediately after injection or sampling, leaving the port exposed to contamination.

Attempts to resolve the issue of having to replace a removed cap have been described, for example, in U.S. Pat. No. 10,328,252, wherein a spring-loaded mechanism is employed to ensure that the cap automatically closes. However, due to the required movement, these spring-loaded caps are limited to covering only the top surface of the injection port and do not provide full enclosure of the top and side surfaces of the port. Accordingly, these spring-loaded caps fail to adequately limit the exposure of the port to microbial contamination.

A vial for containing sterile drug substances, in solid or liquid form, or a vial for containing liquid diluents for such drug substances, has an open top which is typically sealed under sterile conditions using a silicone or rubber stopper held into place by a metal or polymeric covering crimped or molded into position over the stopper. This covering is designed such that a portion of the covering is removed or peeled away to provide access to the stopper disposed thereunder. The stopper can then be punctured by a hypodermic needle to withdraw or fill contents within the vial. However, due to exposure to contaminants when the removable portion of the covering is removed, such vials can be required to be single-use only.

A single-use vial (also termed “single-dose” vial) is a vial of liquid medication intended for parenteral administration (injection or infusion) to a single patient for a single case, procedure, or injection. Single-dose or single-use vials are labeled as such by the manufacturer and typically lack an antimicrobial preservative. When less than the entire contents of the vial are extracted in the first or single use, remaining drug or diluent is wasted.

Against accepted protocol, vials used one time and having remaining drug or diluent therein, are often wiped with disinfectant to remove contaminants, and then re-used (punctured with a hypodermic needle a subsequent time.) This practice can present a risk of infection to patients being administered drugs which are removed in a second or subsequent puncture of the stopper of a single-use vial. There have been multiple outbreaks resulting from healthcare personnel using single-dose or single-use vials for multiple procedures or in more than one patient. Single-use vials must be discarded following its single use because sterility cannot be guaranteed.

Therefore, there is a need for an effective and inexpensive way to provide a cap which can address the deficiencies of the prior art injection port and drug vial covers or caps.

### BRIEF SUMMARY OF THE INVENTION

The present invention relates to a cap assembly affixable to an injection port of an intravenous catheter or IV administration set having a Luer-type fitting for protection against microbial contamination of the injection port. A cap assembly of the invention encloses the top and side surfaces of the injection port sufficiently to protect against and prevent contamination from microbes, and to maintain a sterile field on the outer surface of the Luer-type fitting. The cap assembly preferably comprises a pivoting cover which automatically closes when not in use with a syringe, thereby being capable of wiping, cleaning, disinfecting, or sterilizing an exposed surface of the injection port.

Alternatively, the cap assembly of the invention can be configured to be coupled to a stoppered vial containing sterile drug or diluent. When in a closed position, the cover of the cap assembly prevents microbial contamination of the outer surface the stopper and maintains a sterile field beneath the cover. Advantageously, the cap assembly configured to be coupled to a stoppered vial can convert a single use vial to a multi-use vial, i.e., permitting multiple punctures with a hypodermic needle with lower risk of contamination of the stopper and contents of the vial.

The subject invention further includes a method of using the subject cap assembly for disinfecting or sterilizing a surface on an injection port having Luer-type fitting, or for disinfecting or sterilizing a vial stopper, to prevent microbial contamination or to disinfect or sterilize a microbial-contaminated surface of the vial stopper, thereby maintaining a sterile field during use of the injection port or vial stopper.

In one embodiment of the present invention a cap assembly that encloses a Luer-type fitting of an injection port, termed a “Luer-enclosure cap assembly,” that can disinfect and minimize contamination of an exposed surface of the injection port by providing a pivoting cover that exerts a rubbing action on at least the top surface of the injection port, which can serve to wipe away a microbial contaminant that may be present on that surface.

One embodiment of a cap assembly of the invention comprises a base configured for attachment to and enclosure of top and side surfaces of a Luer-type fitting, and a spring-loaded cover pivotally or hingedly affixed to the base, wherein the cover comprises a disinfecting means, such as a disinfecting implement or device, disposed on an inner surface of the cover. In a preferred embodiment, the disinfecting implement or device is an absorbent or adsorbent pad capable of retaining a disinfecting agent, such as an antimicrobial solution, and dispensing the disinfecting agent onto the surface of the injection port when contacting the surface. Alternatively, the disinfecting implement or device can be a source of ultraviolet (UV) light which disinfects or sterilizes the injection port surface when the UV light contacts the surface of the injection port. It is understood that the UV light source, itself, may not physically contact the surface of the injection port and that exposure of the injection port surface to UV light from the source can achieve the disinfecting or sterilizing result.

In a preferred embodiment of the invention, the cap assembly entirely encloses the side and top surfaces of one end (the top end accessed by a user) of a Luer-type fitting, such as provided on an injection port of an intravenous (IV) administration set. In one embodiment of the invention, a seal is formed between the base and cover when the cover is in a closed position. More preferably, the seal between the cover and base is an airtight seal. In a preferred embodiment, a Luer-type fitting is sealably enclosed by the cap assembly wherein the base sealably couples to an injection port or an injection port extension coupler, and the base and cover sealably engage one another when in the cover is in its closed position. In one preferred embodiment, the cap assembly of the invention comprises a cover hingedly or pivotally connected to the base, and further comprises an injection port extension coupler. In a more preferred embodiment, the cap assembly cover is seated to the cap assembly base in an airtight manner.

In an alternative embodiment, a cap assembly of the invention includes a confirmatory feedback signal that is sensorily communicated to a user, e.g., audibly or by “feel,” when the cap assembly cover being is fully closed and forms an airtight seal with the base.

The cover is preferably pivotally or hingedly affixed to the outer surface of the base using at least one pivot joint or hinge joint, wherein the cover pivots or hingedly moves in a generally arcuate path into an open or closed position. In certain embodiments, the cover is hingedly or pivotally affixed to the outer surface of the base. In an alternative embodiment of the invention, the cover is slidably coupled to the base.

In an alternative embodiment, a cap assembly of the invention comprises a cover having a flange extending outward and away from the outer surface of the cover. The flange is configured to be manipulated by a finger or thumb such that when pressure is applied to the flange, the flange hingedly opens the cover away from the closed position to an open position. When in an opened position, the stopper of the injection port or vial is exposed for use. In a preferred embodiment, the flange extends curvilinearly away from the cover to facilitate ergonomic engagement with the finger or thumb of the user.

In an alternate embodiment, a cap assembly of the invention comprises at least one raised area or rib formed on the base, cap, or base and cap. The raised rib or ribs matingly engage an opposing mated area on the base, cap, or base and cap to resist opening of the cover away from the base without external force being applied.

In a preferred embodiment of the invention, the cover is configured in domed shape. In a preferred embodiment of the invention, the cover is spring-loaded, meaning that it is affixed to the base using a spring, which biases the cover in an open or closed position. It would be understood in the art that the spring-loaded mechanism referred to would include the use of either a compression spring, torsion spring, spiral spring, leaf spring, or any other type of spring or combination thereof. A preferred embodiment comprises a torsion spring, which is biased in the closed position.

Preferably, the spring-loaded cover is biased in the closed position such that a counter force is applied to move the cover from the closed to the opened position, and the cover reverts to the closed position automatically, by force exerted by the spring once the counter force is released. In an embodiment having the cover is slidably coupled to the base, and comprises a disinfecting implement or device configured as an absorbent or adsorbent pad, this automated closing action of the cover can advantageously wipe clean the top surface of the injection port, preventing or reducing the risk of contamination of the injection port.

A further advantage of the cap assembly having a cover comprising a disinfecting pad disposed on the inner surface of the cover is that, when the cap assembly is coupled to an injection port and the cover is in the closed position, the disinfecting pad is in direct contact with the top surface of the injection port valve. In a preferred embodiment, the disinfecting pad is disposed on an inner surface of the cove and completely enclosed by the cover, such that loss of disinfectant, by evaporation or other process, is minimized.

In certain embodiments of the invention, the disinfecting pad material is capable of receiving and storing a disinfectant. The material generally includes adsorptive or absorptive properties which allow the disinfectant to be stored by the pad such that the disinfectant can contact or be delivered to the surface of the injection port. In certain embodiments, a disinfectant can be applied directly to the disinfecting pad as needed. In another embodiment, the disinfecting pad can be infused with a disinfectant and is retained for the life of the cap assembly, wherein disinfectant is not required to be added or reloaded onto the pad.

The disinfecting pad can be a sponge material, gel material, foam material, woven material, non-woven material, and/or a polymeric material such that a disinfectant makes sufficient contact with the surface of the injection port valve to disinfect the surface. Thus, when the cover moves in an arcuate motion from the closed position to the opened position and vice versa, it provides a rubbing motion that advantageously wipes away contaminants and applies the disinfectant.

In another embodiment of the invention, the disinfecting implement or device comprises an ultraviolet (UV) cell disposed on the inner surface of the cover to expose the injection port surface to a disinfecting or sterilizing UV light. The UV cell can remain on at all times or, preferably, comprises a switch or timer to activate and inactivate (turn ON and OFF) the UV cell. For example, a switch can be incorporated into the cap assembly to manually activate and inactivate the UV cell. Alternatively, the switch can be a motion sensor or can be activated by closing of the cover. Opening the cover can inactivate the UV cell. Alternatively, a timer can be activated manually or by movement of the cover such that the UV cell is inactivated for a period of time or is inactivated after a set period of time. In a more preferred embodiment, the inner surface of the cover contains both an absorbent pad containing a disinfectant and a UV-light source.

It would be understood in the art that a cap assembly of the present invention can be coupled to any port, such as provided on a vial or other container, having a Luer-type fitting and wherein the port is advantageously protected from contamination or in need of disinfecting or cleansing such as a vial or other container.

In addition, it is understood that the cap assembly can be integrally formed as part of the injection port, per se, thereby forming an IV administration set having an injection port wherein the injection port comprises a cap assembly as described.

In an alternative embodiment, a cap assembly according to the invention comprises the cover and base as described above, but where the base is configured at one end (the bottom end) to matingly couple to a stoppered vial. These vials are well known in the art and are described herein for containing sterile drug compositions or diluents.

The subject invention further comprises a method for protecting an injection port having a Luer-type fitting, or a puncturable surface of stoppered vial using a cap assembly as described herein to prevent or ameliorate microbial contamination or maintain a sterile field on the outer, user-accessible surface of the Luer-type fitting or vial stopper.

A method of the invention useful for protecting or preventing contamination of a Luer-type fitting can comprise the steps of:
a) providing a cap assembly of the invention configured for coupling to a Luer-type fitting;
b) coupling the cap assembly to an injection port having a Luer-type fitting, wherein the cap assembly includes a cover having a disinfectant means disposed on its inner surface;
c) moving the cover into its open position to expose the injection port and injection port valve;
d) connecting a needleless syringe having a Luer-type fitting to the injection port for delivery or retrieval of a fluid through the injection port;
e) disconnecting the syringe from the injection port; and
f) allowing the cover to spring-loadingly return to the closed position thereby contacting the outer surface of the injection port with disinfectant and retaining disinfectant in contact with the injection port and injection port valve while in the closed position.

A method of the invention useful for protecting or preventing contamination of a stoppered vial can comprise the steps of:
a) providing a cap assembly of the invention configured for coupling to a stoppered vial containing sterile drug or diluent;
b) coupling the cap assembly to the stoppered vial, wherein the cap assembly includes a cover having a disinfectant means disposed on its inner surface;
c) moving the cover into its open position to expose a puncturable top surface of a stopper disposed in the stoppered vial;
d) puncturing the stopper with a hypodermic needle connected to a syringe for delivery or retrieval of a fluid through the stopper;
e) withdrawing the hypodermic needle and syringe from the stoppered vial; and
f) allowing the cover to spring-loadingly return to the closed position thereby contacting the outer surface of the stopper with disinfectant and retaining disinfectant in contact with the stopper while in the closed position.

In a method employing a cover having UV cell for disinfecting or sterilizing the surface of the injection port, the steps are:
a) providing a cap assembly of the invention comprising a cover having a UV cell disposed on its inner surface;
b) coupling the cap assembly to an injection port having a Luer-type fitting or to a stoppered vial containing a sterile drug or diluent;
c) moving the cover into its open position and connecting of a syringe to the injection port for delivery or retrieval of a fluid through the injection port or puncturing the vial stopper with a hypodermic needle to fill or withdraw contents from the vial;
d) disconnecting the syringe from the injection port or withdrawing the needle from the vial stopper; and
e) allowing the cover to spring-loadingly return to the closed position thereby exposing the top surface of the injection port and injection port valve to UV light while the cover is in the closed position.

In certain embodiments, allowing the cover to return to the closed position (step e)) can activate a switch or timer, thereby activating the UV light to expose the top surface of the injection port and injection port valve to disinfect or sterilize the injection port surface.

In an embodiment using a cap assembly having a disinfectant pad where disinfectant can be added, a method of the invention can further comprise the step of adding disinfectant to the disinfectant pad as needed.

In another embodiment, the method can comprise coupling the cap assembly to a medical vial or container having a Luer-type fitting or having diaphragm penetrable by a needle having a Luer-type fitting.

The subject invention further encompasses a method for converting a single-use vial containing sterile drug or diluent into a multi-use vial containing sterile drug or diluent using a cap assembly. Such method comprises the steps of:
a) providing a cap assembly of the invention;
b) opening and using the single-use vial a first time;
c) coupling the cap assembly to the single-use vial over a puncturable stopper provided on the single-use vial, wherein the cap assembly can be opened and closed a plurality of times for use as needed until the vial no longer contains a useful amount of sterile drug or diluent, and
d) closing the cover of the cap assembly following each use to prevent contamination and maintain a sterile field for the stopper.

In addition, the subject invention also envisions a kit comprising the cap assembly of the present invention and an intravenous administration set having a Luer-type fitting injection port.

Thus, the subject invention provides an advantageous and inexpensive device and method for protecting and disinfecting an injection port having a Luer-type fitting, or a stoppered end of a vial containing injectable drug or diluent. In certain embodiments, the cap assembly can provide a rubbing action to clean and disinfectant the top surface of the injection port and injection port valve or stoppered vial. Other advantages and applications of a device or system of the invention will be recognized and understood by persons of ordinary skill in the art in view of the detailed description and drawings provided herewith.

### BRIEF DESCRIPTION OF THE SEVERAL VIEWS OF THE DRAWINGS

The features and advantages of the invention will be readily understood by reference to specific embodiments which are illustrated in the appended drawings. These drawings depict only typical embodiments of the invention and are not to be considered as limiting the scope of the invention.

FIG. 1 is a side view of an embodiment of a cap assembly device according to the subject invention illustrating a cover, shown in closed position, pivotally connected to a base which facilitates the seating of the pivoting cover The base is shown to include or adjoin an extension coupler having a Luer-type fitting for matingly connecting with a needleless syringe (shown) at one end (the top end, accessed by a user) of the extension connector and matingly connecting with an injection port of an IV administration set (not shown) at the other end (the bottom end connected to the injection port) of the extension connector.

FIG. 2 is a perspective view of an embodiment of a cap assembly device according to the subject invention, as shown in FIG. 1, illustrating a Luer-type fitting accessible when the cover is in open position.

FIG. 3 is a cross-section side view of an embodiment of a cap assembly device having a cover in open position as in FIG. 2, and illustrating the cap assembly device, as coupled to a syringe having a Luer-type fitting.

FIG. 4 is a side view (not in cross-section) of an embodiment of a cap assembly device having a cover in open position, as in FIGS. 2 and 3, and coupled to a syringe via a Luer-type fitting.

FIG. 5 is a side view (not in cross-section) of an embodiment of a cap assembly device according to the subject invention having a cover in open position, as in FIGS. 2, 3, and 4 wherein the cap assembly is uncoupled from a syringe having a Luer-type fitting.

FIG. 6 shows elevational views A-D of an embodiment of a cap assembly according to the subject invention, as configured for coupling to a Luer-type fitting on an injection port of an intravenous administration set: view A is a side view showing a curvilinear (“trigger-shaped”) lever, or “trigger lever” on the left side of the cap assembly; view B is a front view showing the trigger lever on the back side of the cap assembly; view C is a side view showing the trigger lever on the right side of the cap assembly; and view D is a back view showing the trigger lever on the back side of the cap assembly.

FIG. 7 is a cross-sectional view of the cap assembly embodiment shown in in FIG. 6, view A, illustrating certain inner components not viewable from the views in FIG. 6, including the presence of a torsion spring for spring-loaded action of the cover, a disinfecting means disposed on the inner surface of the cover, and an O-ring disposed around the periphery of the contacting faces of the cover and base.

FIG. 8 is a cross-sectional view of the cap assembly embodiment shown in in FIG. 6, view C, illustrating the cover in the open position and in relation to its use with a syringe having a Luer-type fitting.

FIG. 9 shows an elevational side view of an alternative embodiment of a cap assembly according to the subject invention, wherein the cap assembly is configured or adapted for coupling to a stoppered vial and does not require, and does not include as shown, a Luer-type fitting or extension coupler component.

FIG. 10 shows elevational views of the embodiment of a cap assembly shown in FIG. 9, configured for coupling to a stoppered vial: view A is a left-side view showing the trigger lever on the right side of the cap assembly; view B is a back view showing the trigger lever on the back side of the cap assembly; view C is a right-side view showing the trigger lever on the left side of the cap assembly; and view D is a front view showing the trigger lever on the back side of the cap assembly.

FIG. 11 is a perspective view of a cap assembly according to an embodiment of the invention adapted for protecting a stoppered vial, wherein the cap assembly is connected to a stoppered vial and the cover of the cap assembly is shown in the open position.

FIG. 12 is a perspective view of a cap assembly as shown in FIG. 11 coupled to a stoppered vial, wherein the vial is shown in cross-section to illustrate the orientational relationship of the cap assembly and vial stopper when coupled or connected to one another.

FIG. 13 is a perspective view of a cap assembly according to an embodiment of the invention, as shown in FIG. 10 and FIG. 11, illustrating the orientational relation of the cap assembly with a syringe and hypodermic needle, shown punctured through the vial stopper, in use.

FIG. 14 illustrates components of a kit comprising an IV administration set (A) and a cap assembly (B) configured for connecting to an injection port on the IV administration set.

### DETAILED DESCRIPTION OF THE INVENTION

The subject invention can be understood by the description provided herein, along with reference to the accompanying drawings. It will be readily understood that the components of the present invention, as generally described and illustrated in the figures herein, can be arranged and designed in a wide variety of different configurations within the scope of the invention described. Thus, the following detailed description, as represented in the figures, is not intended to limit the scope of the invention as claimed but is merely representative of presently preferred embodiments of the invention.

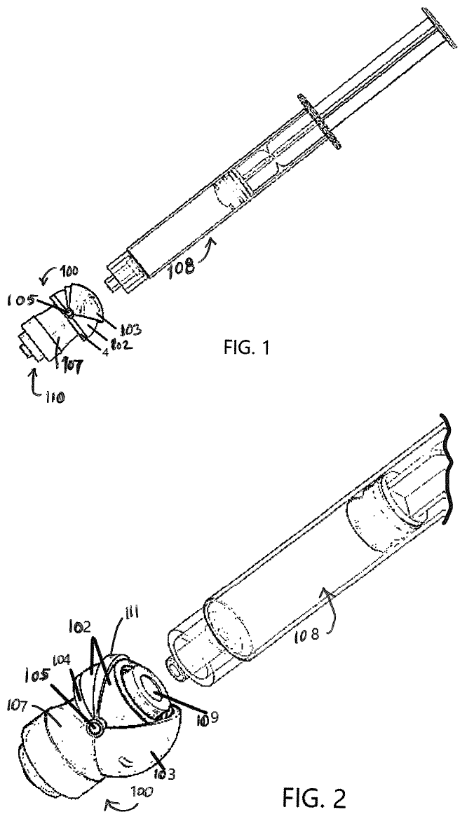

Referring to FIG. 1, an embodiment of the disinfecting cap assembly 100 device of the invention is shown in a side view illustrating elements of the invention, namely, a base 102 and extension coupler 107 having a first end (a bottom end) 110 adapted for coupling to a Luer-type fitting of an injection port, and a second end (a top end accessed by a user; detail not shown) adapted for coupling to Luer-type fitting on a (needleless) syringe. Cover 103 is shown in FIG. 1 in a closed position, the cover being pivotally affixed to the base at hinge joint or pivot joint 105. Preferably, the hinge joint or pivot joint 105 is spring-loaded. As shown, the cap assembly 100 is adjoined to an extension coupler 107 having a Luer-type fitting at each end. The first end of extension coupler 107 is adapted for coupling to a Luer-type fitting provided on an injection port of an IV administration set. The second end of extension coupler 107 is adapted to matingly engage and receive a needleless syringe having a Luer-type fitting. As shown, the base 102 is substantially cylindrical and annularly engages with the extension coupler 107; however, it would be understood in the art that other shapes of the base can be formed without detracting from the functionality of the invention.

FIG. 1 also illustrates cover 103, which is preferably spring-loaded and pivotally affixed to the outer face of the base 102 in a manner that allows the cover to be biased into the closed position. Cover 103 is spatially oriented in relation to base 102 such that when force is applied, it is allowed to move in an arcuate motion from a closed position to an opened position, exposing the top surface extension coupler 107. In an alternative embodiment, the cover is spring-loaded and hingedly affixed to the outer face of the base 102 in a manner that allows the cover to be biased into the closed position while allowing it to move in an arcuate motion from a closed position to an opened position. It would be understood in the art that the spring-loaded mechanism referred to would include the use of either a compression spring, torsion spring, spiral spring, leaf spring, or any other type of spring or combination thereof.

However, it would be understood in the art that the cover may also be affixed to the inner surface of the base while still allowing the cover to be spring-loaded and to move in an arcuate motion. The cover 103 component preferably has a curved outer surface or is dome-shaped wherein the curve is shaped downwardly and towards the base. The preferred dome shape of the cover 103 is not required or critical. For example, the top outer surface of cover 103 can be flat or angular.

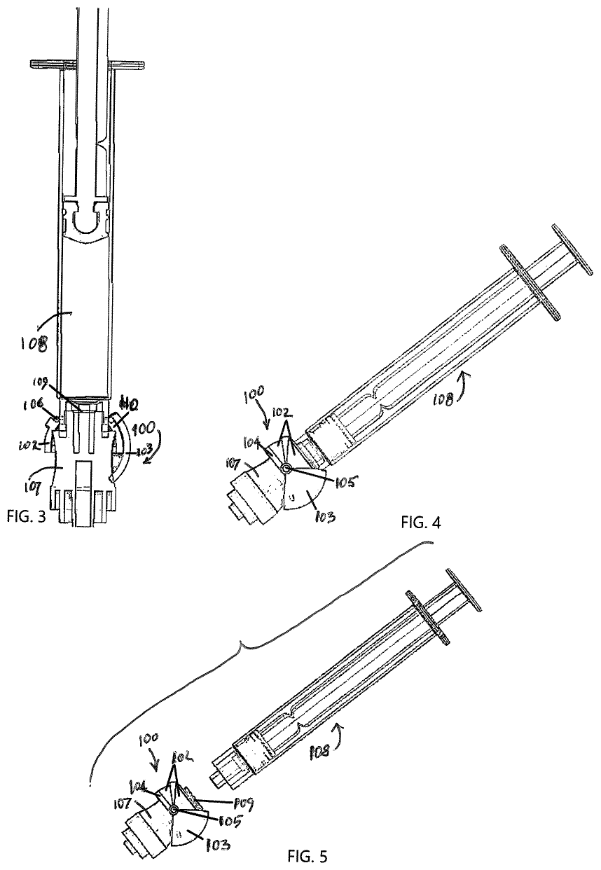

Cover 103 has an inner face, wherein a disinfecting means, such as a disinfecting implement or device (depicted in FIG. 3), can be disposed. In the embodiment shown, the disinfecting means is preferably an absorbent or adsorbent pad disposed proximate to the inner surface of cover 103, wherein the pad is positioned to contact and wipe the top surface of the extension coupler when the cover moves from an open position to the closed position, and vice versa.

Alternatively, the disinfecting implement or device can be an ultraviolet (UV) light source or UV cell providing disinfecting or sterilizing UV light to the top surface of the extension coupler. Advantageously, a UV cell serving as the disinfecting implement or device can disinfect or sterilize the target surface without physically contacting the injection port.

A disinfecting implement or device configured as an absorbent or adsorbent pad is enclosed by the cover 103. The preferred disinfecting pad material can have adsorptive or absorptive properties which allow a fluid disinfectant to be stored within the material. Disinfectant can be added or reloaded onto the pad by applying disinfectant directly to the disinfecting pad as needed. In a preferred embodiment, the disinfecting pad is provided infused with a disinfectant such that the amount of disinfectant remains sufficient for use of the life of the cap assembly and does not require added disinfectant or reloading of disinfectant onto the pad.

The disinfecting pad can comprise any suitable absorbent or adsorbent material, for example, a sponge material, gel material, foam material, woven material, non-woven material, and/or a polymeric material such that the contained disinfectant is released or contacts the proximate contacting surface, such as the top surface of the extension coupler, when the cover is in a closed position. Advantageously, in the embodiment shown, moving cover 103 in an arcuate motion from the closed position to the opened position, or from the closed position to the opened position, the pad contacts the extension coupler in a rubbing motion that wipes away contaminants and applies the disinfectant to the top surface of the extension coupler. Commonly used disinfectants are solutions containing alcohols (ethanol, methanol, propanol, isopropanol, or the like) iodine or iodine mixtures with alcohols, polyvinylpyrrolidone, or other available disinfectants.

FIG. 2 shows a perspective view of an embodiment of a disinfecting cap assembly 100 according to one embodiment of the subject invention, in the opened position and illustrating a base 102 capable of coupling at one end 104 to an extension coupler 107, which in turn can be connected to an injection port of an IV administration set having a Luer-type fitting. Cover 103 is pivotally affixed to the base 102 by a spring-loaded pivot joint or hinge joint 105. Cover 103 is shown in FIG. 2 in its open position, exposing the top surface 109 of the extension coupler.

As shown, a needleless syringe 108 having a Luer-type fitting can be coupled to the cap assembly. Illustrated in FIG. 2 is a stop 111 which blocks the movement of the cover, and which serves to retain the cover in a position that encloses the top surface of the injection port when the cover is in the closed position. The stop 111 shown in FIG. 2 is shown as a curved portion semi-annularly surrounding one side of the base. However, it would be understood that the stop can be any protrusion from the side of the base that prevents the cover from moving past its closed position. For example, one or more knobs or nubs can serve as a stop for the arcuately moving cover.

FIG. 3 is a side view of an embodiment of a cap assembly 100 device according to the subject invention, shown in cross-section, comprising base 102, and cover 103 in the opened position to expose the top surface 109 of the cap assembly for receiving and engaged with needleless syringe 108, having a male Luer-type fitting. Depicted in FIG. 3 is disinfecting means 110, which as shown can be an absorbent pad for retaining a disinfectant. As shown, the base 102 comprises an O-ring 106 disposed on its exposed surface when cover 103 is in an open position, which can seal the connection of the cap assembly with the engaged needleless syringe. It would be understood in the art that O-ring 106 referred to in the illustration could comprise silicon, rubber, or other material known in the art for sealably connecting a Luer-type fitting.

FIG. 4 is a side view of an embodiment of a cap assembly 100, and is the non-cross-sectional view of FIG. 3. Base 102 has a first, bottom end 104 which adjoins extension coupler 107, and a second, top end 109 which is exposed when cover 103 is in the opened position, to engage and couple with syringe 108 having a Luer-type fitting. Cover 103 is pivotally connected to the base 102 at pivot joint or hinge joint 105.

An exploded view of FIG. 4, where the syringe 108 is shown separated from cap assembly 100, is shown in FIG. 5. FIG. 5 illustrates a side view of cap assembly 100 showing cover 102 in the opened position, exposing top surface 109 which is covered and disinfectingly protected when the cover 103 is in the closed position.

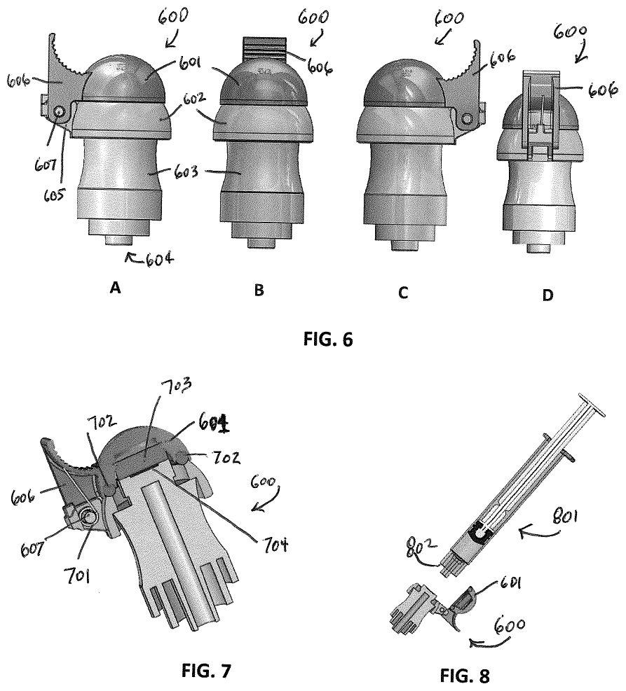

Another embodiment of the invention is depicted in FIGS. 6-8. FIG. 6 shows the right side, front, left side, and back of one embodiment of a cap assembly 600 in elevational views A-D, respectively. Being generally cylindrical, the orientation of front and back, and right and left sides of the cap assembly of the invention are relative, and are arbitrarily assigned by designating hinge joint 607 to be positioned on the “back” of the cap assembly.

In this embodiment, the first or bottom end of base 602 is configured for coupling to a Luer-type fitting on an injection port of an intravenous administration set. For ease of adapting and connecting a cap assembly of the invention to an injection port, extension coupler 603 is affixed to the bottom end of base 602. Extension coupler 603 is configured at one end 604 to engage a Luer-type fitting on an injection port and configured at its opposite end to receive a Luer-fitting on a needleless syringe. Base 602 is annularly disposed in relation to extension coupler 604 and engages at its opposite (top) end with cover 601. Annular base 602 comprises flange 605 forming a first portion of a hinge joint.

Cover 601 comprises flange 606 forming a second portion of a hinge joint, matingly receiving and adjoined to base flange 605 by hinge post 607 disposed within mated channels formed within the hinge portions of flanges 605 and 606. The channels and hinge post are aligned perpendicular to the longitudinal midline of the cap assembly and extension coupler.

Base flange 605 and cover flange 606 matingly engage with hinge post 607 and with one another to form a hinge joint about which the cover 601 hingedly opens and closes. Base flange 605 can preferably be integrally formed or molded as part of the base 602, or can be formed separately and affixed thereto. Cover flange 606 can preferably be integrally formed or molded as part of the cover 601, or can be formed separately and affixed thereto.

Cover flange 606 is preferably configured in an ergonomic shape to facilitate manual actuation, and preferably actuation using one hand. More preferably cover flange 606 is configured to facilitate actuation of cover 601 using one manual digit, e.g., finger or thumb, of the user. Effectively, cover flange 606 serves as a lever for manually opening the cover 601 for access to the top end of the injection port or extension coupler, where hinge post 607 serves as the fulcrum for the lever, providing mechanical advantage and facilitating the opening of cover 601 by using one hand, preferably one finger or thumb of the user. More preferably, as shown, cover flange 606 can have a portion formed in a curvilinear shape, e.g., in a shape akin to a trigger and therefore referred to herein as a “trigger lever,” which can further facilitate actuation using a thumb. View A in FIG. 6 is a right-side view showing the trigger lever on the left side of the cap assembly; view B is a front view showing the trigger lever on the back side of the cap assembly; view C is a left-side view showing the trigger lever on the right side of the cap assembly; and view D is a back view showing the trigger lever facing the viewer on the back side of the cap assembly.

FIG. 7 is a cross-sectional view of the cap assembly embodiment 600 shown in in FIG. 6, illustrating a torsion spring 701 engaged with hinge post 607 and trigger lever 606, wherein the torsion spring provides resistance for the trigger lever, whereby the resistance force of torsion spring 701 maintains bias of cover 601 in the closed position. Also illustrated in FIG. 7 is O-ring gasket 702 disposed between, and at the entire interface of, base 602 and cover 601. The spring-loaded bias of cover 601 in the closed position provided by torsion spring 701 can provide an airtight seal between base 602 and cover 601 by applied pressure to O-ring gasket 702. It has been found that O-ring gasket is preferably disposed or recessed circumferentially on cover 601, on a face contacting base 602.

Further illustrated in FIG. 7 is a disinfecting means 703 disposed on an inner face of cover 601. For illustration purposes, disinfecting means is shown as an absorbent pad having disinfecting agent absorbed therein for contacting, cleaning, or disinfecting the Luer-type fitting surface 704 where a syringe having a Luer-type fitting is connected for administering or withdrawing fluid. Advantageously, the spring-loaded bias of cover 601 in the closed position provided by torsion spring 701 can maintain contact between an absorbent pad disinfecting means 703 and Luer-type fitting surface 704 when the cover 601 is in the closed position.

Alternatively, it would be understood that disinfecting means 703 can be an ultraviolet (UV)-light source or UV-light cell which provides disinfecting properties at UV wavelengths. As would be understood, contact between disinfecting means 703 and needle-puncture surface 704 is not critical using a UV disinfecting means, whereby UV light can disinfect without physical contact between the source and the target surface being disinfected by the UV light.

FIG. 8 is a cross-sectional view of the embodiment of cap assembly 600, as shown in cross-section in in FIG. 7, illustrating cover 601 in the open position, and in relation to its use with a syringe 801 having a Luer-type fitting 802.

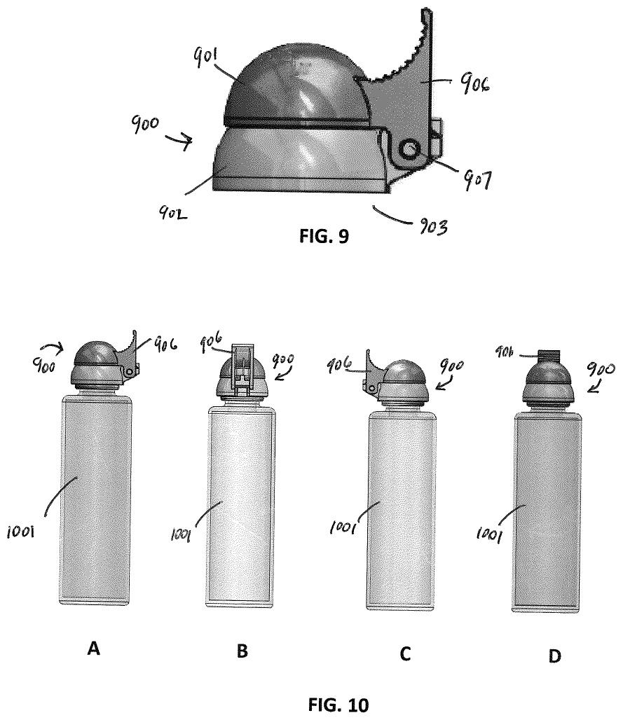

FIG. 9 shows an alternative embodiment of a cap assembly 900 according to the subject invention, configured for coupling to a stoppered vial (not shown) wherein the stopper is penetrable or puncturable with a hypodermic needle to access and withdraw or fill contents of the vial, e.g., liquid drug substance or diluent contained with the chamber of the vial. This embodiment is related to the embodiment shown in FIG. 6, having a flanged cover 901 hingedly affixed to flanged base 902 by hinge post 907, but wherein base 902 is configured at its bottom end 903 to be coupled to a stoppered vial.

Elevational views of the left side, back, right side, and front of cap assembly embodiment 900 are shown in FIG. 10, views A-D, respectively, coupled to a stoppered vial 1001 having a stopper penetrable or puncturable with a hypodermic needle to access contents, e.g., liquid drug substance or diluent contained with the chamber of the vial. Being generally cylindrical, the orientation of front and back, and right and left sides of this embodiment of cap assembly 900 of the invention are relative, and are arbitrarily assigned by designating hinge joint post 907 to be positioned on the “back” of the cap assembly.

View A is a side view showing trigger lever 906 on the right side of the cap assembly 900; view B is a back view showing trigger lever 906 on cap assembly 900 facing the viewer; view C is a side view showing trigger lever 906 on the left side of the cap assembly 900; and view D is a front view showing the trigger lever 906 on the back side of the cap assembly 900.

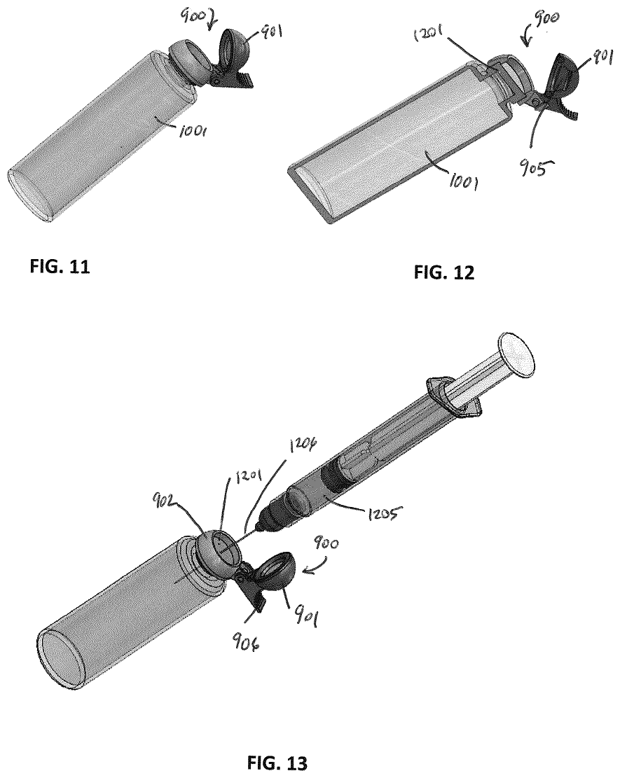

FIG. 11 is a perspective view of a cap assembly 900 according to an embodiment of the invention, wherein the cap assembly and cover 901 (shown in the open position) are coupled to a stoppered vial 1001 (stopper not shown).

FIG. 12 is a perspective view of a cap assembly according to an embodiment of the invention, wherein the cap assembly 900 and cover 901 (shown in the open position) are coupled to a stoppered vial 1001 shown in cross-section to illustrate the vial stopper 1201 and disinfectant means 905 disposed on an inner surface of cover 901.

FIG. 13 is a perspective view of a cap assembly 900 according to an embodiment of the invention, as shown in FIG. 10, at a slightly different viewing angle (toward the viewer) to show vial stopper 1201 and to further illustrate the cap assembly 900 and cover 901 (shown in the open position) in relation to its use with a syringe 1205 and hypodermic needle 1206 shown punctured through the vial stopper 1201.

In a method of the invention a cap assembly suitable for connecting to an IV administration set or a stoppered vial, i.e., as described herein can be provided, with or without an extension coupler, and coupled to the IV administration set or stoppered vial, wherein the cap assembly has the cover in a closed position. In the closed position, disinfecting implement or device can disinfect a top surface of the injection port or stoppered vial. A cover component of the cap assembly is moved in an open position, using one hand, and preferably one finger or thumb of one hand to access the injection port or vial stopper for filling or withdrawing contents therefrom. Following filling or withdrawing contents, the cover is released and allowed to spring into a closed position, thereby protecting the top surface of the coupled injection port or stoppered vial and maintaining a sterile field on that top surface.

In one embodiment, moving the cover in an arcuate manner about its one or more pivot joints and into its open position, the cover can be moved in an arcuate path, thereby wiping disinfectant across the top surface of the injection port and injection port valve.

As one example, a syringe or other medical apparatus having a Luer-type fitting can be connected to the injection port with the cap assembly in the opened position to administer a fluid to the patient or to withdraw a sample from the patient. Upon completion of the administration or withdrawal of fluid to or from the vessel, the syringe can be disconnected and removed from the injection port, thereby allowing the spring-loaded cover to automatically return to the closed position. Advantageously, when the cover returns to the closed position, disinfecting implement or device, when provided as an absorbent or adsorbent pad again wipes clean the top surface of the injection port and injection port valve, and retains the disinfectant pad in contact with the top surface of the injection port and injection port valve, and encompasses the entire top portion of the injection port to facilitate a clean and microbe-free surface.

In an embodiment comprising a UV light source or UV cell as a disinfecting implement or device, returning the cover to the closed position positions the UV cell over the surface of the injection port such that the surface of the injection port is exposed to disinfecting or sterilizing UV light, thereby disinfecting or sterilizing the surface of the injection port without the injection port and UV cell physically contacting one another.

It would be understood that the cap assembly may be connected to medical vials or other devices having a Luer-type fitting where these disinfecting procedures are preferred.

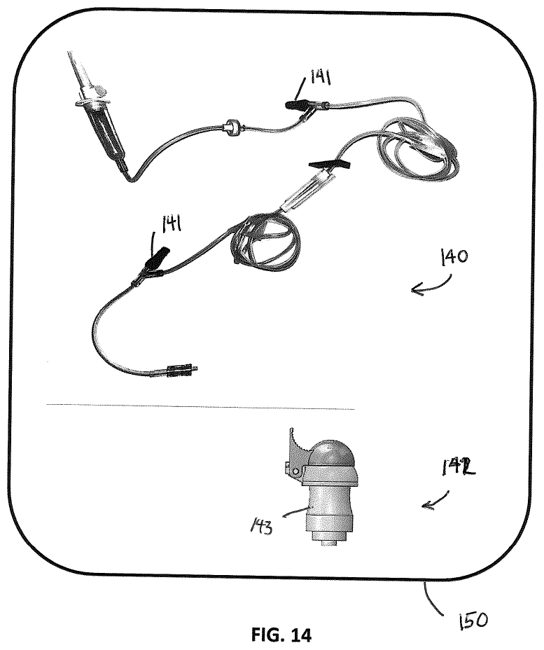

It would also be understood that the invention can include a kit comprising an IV administration set having an injection port with a Luer-type fitting, and a cap assembly as descried herein for use with the IV administration set, packaged together. An embodiment of a kit according to the subject invention is illustrated in FIG. 14, which depicts an IV administration set 140 having at least one injection port 141 provided with a Luer-type fitting, and a cap assembly 142. Shown is cap assembly embodiment 600 described herein for illustration purposes only. A cap assembly included with a kit of the invention can optionally include extension coupler 143.

Alternatively, an IV administration set may be provided having an injection port having a cap assembly formed integrally therewith, wherein the injection port is manufactured to include a pivoting or hinged cap assembly as a single unit.

Written instruction for use of the cap assembly and IV administration set may also be included in the kit having a separately provided cap assembly or having an injection port comprising an integrally formed cap assembly.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

The above disclosure and example generally describe the present invention and is provided for purposes of illustration and is not intended to limit the scope of the invention. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A cap assembly capable of disinfectingly enclosing a Luer-type fitting of an injection port of an intravenous administration set or capable of enclosing a hypodermic needle-puncturable stopper on a single-dose stoppered vial containing sterile drug or diluent, said cap assembly comprising:
a base having a first or bottom end configured to couple to the injection port or the stoppered vial and a second or top end configured to receive cover,
a cover having an outer surface and an inner surface, said cover being moveable between an open position and a closed position, and
a disinfecting means disposed on the inner surface of the cover, wherein the disinfectant means is selected from an absorbent pad or adsorbent pad retaining a disinfectant, and an ultraviolet (UV)-light source,
wherein said base and cover are hingedly or pivotally affixed to one another wherein said hinge or pivot is spring-loaded such that the cover is biased in the closed position to form a self-closing cover.

2. The cap assembly of claim 1 wherein the cap assembly is separate from and connectable to the Luer-type fitting provided on the injection port of an intravenous administration set.

3. The cap assembly of claim 1 wherein the cap assembly is separate from and connectable to the single-use vial containing drug or diluent.

4. The cap assembly of claim 1 wherein the cap assembly is formed integrally with and part of the injection port of an intravenous administration set.

5. The cap assembly of claim 1 wherein the cover, in its closed position, forms an airtight seal with the injection port or drug or diluent vial stopper.

6. The cap assembly of claim 1 wherein the cover comprises a flange extending outwardly and away from the outer surface of the cover wherein the flange is configured for engagement by a finger or thumb of a user such that applied pressure to the flange hingedly opens the cover away from the closed position to an open position, exposing the stopper of the injection port or vial.

7. The cap assembly of claim 6, wherein the flange extends curvilinearly away from the cover to facilitate ergonomic engagement with the finger or thumb of the user.

8. The cap assembly of claim 1, wherein the cap has a domed outer surface.

9. The cap assembly of claim 1 wherein, upon closing of the cover to form an airtight seal, a confirmatory feedback signal is audibly communicated to a user.

10. The cap assembly of claim 1 wherein the cap assembly comprises a least one raised area or rib formed on the base, cap, or base and cap, which matingly engages an opposing area on the base, cap, or base and cap to resist opening of the cover away from the base without external force being applied.

11. The cap assembly of claim 1, wherein the cap assembly is capable of maintaining a sterile field on the Luer-type fitting or vial stopper, when said cap assembly is in the closed position.

12. The cap assembly of claim 1 wherein said disinfecting means is an absorbent pad containing a disinfectant.

13. The cap assembly of claim 1, wherein said disinfecting means is a UV-light source or UV light cell.

14. The cap assembly of claim 1 wherein said disinfecting means is an absorbent pad containing a disinfectant and a UV-light source.

15. A method for protecting from microbial contamination and maintaining a sterile field in a Luer-type fitting of an injection port on an intravenous administration set or on a stopper on a single-use vial containing a sterile drug or diluent, said method comprising:
providing a cap assembly of claim 1;
coupling the cap assembly to an injection port having a Luer-type fitting or a single-use vial;
opening the cover with one hand to access the injection port or stopper of a single-use vial;
connecting a syringe to the injection port or puncturing the vial stopper with a hypodermic needle for delivery or retrieval of a fluid therethrough;
disconnecting the syringe or needle from the injection port or vial stopper; and
allowing the cover to spring-loadingly return to the closed position thereby exposing said injection port or vial stopper to said disinfectant means.

16. A method for converting a single-use vial containing sterile drug or diluent into a multi-use vial containing sterile drug or diluent, said method comprising:
providing a cap assembly of claim 1;
opening and using the single-use vial a first time;
coupling the cap assembly to the single-use vial over a puncturable stopper provided on the single-use vial, wherein the cap assembly can be opened and closed a plurality of times for use as needed until the vial no longer contains a useful amount of sterile drug or diluent, and
closing the cover of the cap assembly following each use to prevent contamination and maintain a sterile field for the stopper.

17. A kit comprising a cap assembly of claim 1 and at least one of an intravenous administration set having an injection port with a Luer-type fitting or a single-use vial containing sterile drug or diluent.
